(19) United States
(12) Patent Application Publication
(10) Pub. No.: US 2026/0207679 A1
(43) Pub. Date: Jul. 23, 2026

(54) POLLEN-BASED COMPOSITIONS AND EXTRACTS FOR STIMULATING HYALURONIC ACID PRODUCTION

(71) Applicant: WEIR SCIENCE LIMITED, Auckland (NZ)

(72) Inventor: Iona Elizabeth WEIR, Auckland (NZ)

(73) Assignee: WEIR SCIENCE LIMITED, Auckland (NZ)

(21) Appl. No.: 19/134,603

(22) PCT Filed: Dec. 1, 2023
(86) PCT No.: PCT/NZ2023/050143

§ 371 Date: May 30, 2025

(30) Foreign Application Priority Data
Dec. 2, 2022 (NZ) 795225

(51) Int. Cl. A61K 35/644; A61K 47/44; A61K 47/46

(52) U.S. Cl. CPC A61K 35/644; A61K 47/44; A61K 47/46

(57) ABSTRACT

The invention relates to a pollen-based composition, or an extract derived therefrom, wherein the composition or extract stimulates the production of hyaluronic acid (HA). The composition or extract comprises at least glucose-6-phosphate, as a substrate or precursor for the production of HA. The composition or extract of the invention also comprises other small molecular weight bioactive molecules and/or compounds selected from the group comprising flavonoids, flavonols, flavones, alkaloids, aglycones, glycosides, lipids, fatty acids, amino acids, anthranilic acid and isomers thereof, spermidine and isomers thereof, caffeoyl spermidine compounds, and hydroxycinnamic acids. The invention also relates to formulations comprising the pollen-based composition or extract which are useful for the treatment of a variety of conditions including skin conditions and skin aging, and symptoms of Genitourinary Syndrome of Menopause (GSM), including urinary incontinence, vaginal dryness, vaginal atrophy, irritation, itching, cracking, thinning, redness, inflammation and declining libido, sexual function, sexual response and/or pleasure.

### FIELD OF THE INVENTION

The invention relates to pollen-based compositions or extracts, and pharmaceutical and cosmetic formulations or compositions containing same. The compositions and extracts of the invention are useful for treating and preventing skin related conditions, in particular improving skin moisture content, increasing skin firmness and elasticity, improving the function of the skin barrier, improving skin healing, treating skin atrophy including vaginal atrophy, rebalancing skin microbiome, and treating or preventing skin aging. They are also useful for preventing and treating the symptoms of GSM, including urinary incontinence, vaginal dryness, vaginal atrophy, irritation, itching, cracking, thinning, redness, inflammation and declining libido, sexual function, sexual response and/or pleasure.

### BACKGROUND

Human skin aging is a complex biological process, caused by both intrinsic (innate) and extrinsic (external) factors. Intrinsic skin aging is influenced by hormonal changes that occur with age, and it is well established that decreased production of estrogens and androgens results in collagen degradation, skin dryness, loss of elasticity, wrinkling of the skin, epidermal atrophy, and genitourinary syndrome of menopause (GSM) (also known as vaginal atrophy or vulvovaginal atrophy). Extrinsic aging is the result of exposure to external environmental factors, namely ultraviolet (UV) radiation.

Photoaging, general aging and skin atrophy, in particular GSM, share the following hallmarks: a decrease in collagen, elastin and hyaluronic acid which affects skin structure and hydration.

In relation to GSM, a loss of hyaluronic acid in the vaginal tissues contributes to many of the symptoms of GSM, including urinary incontinence, vaginal dryness, vaginal atrophy, irritation, itching, cracking, thinning, redness, inflammation and declining libido, sexual function, sexual response and/or pleasure.

One of the key molecules involved in skin hydration is hyaluronan or hyaluronic acid (HA), a non-sulphated glycosaminoglycan (GAG) with a unique capacity to bind and retain water molecules. HA is widely distributed in connective tissue, epithelial tissue, skin tissue and nerve tissue, and is a major component of the extracellular matrix. At least 50% of HA present in the human body is distributed in the skin, namely in the gap between epidermal cells and the outer matrix of dermal cells.

HA is also present in all tissues and fluids of the body, including in the eye, in synovial fluid, in skeletal tissues, heart valves, the lung, the aorta, the prostate, tissues of the penis and vaginal tissues. HA has numerous functions in the body including hydration, lubrication of joints, regulation of tissue repair and wound healing, activation of inflammatory cells to enhance immune response, providing the framework for blood vessel formation and fibroblast migration.

HA is synthesized by specific membrane bound enzymes called HA synthases (HAS). These enzymes synthesize HA on the inner surface of the plasma membrane and then HA is extruded through pore-like structures into the extracellular space. There are three mammalian enzymes HAS-1, -2 and -3, which exhibit distinct enzymatic properties and synthesize HA chains of various lengths.

It is known that HA in the skin depletes over time, and is therefore a major cause of skin aging, since the epidermis loses the principal molecule responsible for binding and retaining water molecules, resulting in loss of skin moisture. In the dermis, the major age-related change is the increasing avidity of HA with tissue structures with the concomitant loss of HA extractability. This parallels the progressive cross-linking of collagen and the steady loss of collagen extractability with age. This all contributes to the dehydration, atrophy and loss of elasticity that characterizes aged skin and body tissues, including vaginal tissues.

There has been significant research in the last decade into the identification of methods to increase HA production in the skin to improve the condition of the skin and combat signs of aging. These methods include development of injectable dermal fillers comprising cross-linked HA, as well as topical skin formulations comprising HA molecules. While injectable treatments are becoming increasingly popular, such treatments are invasive and expensive, and have the potential for adverse side effects. Topical HA formulations are generally less effective than injectable formulations but offer the advantage of being less invasive, less expensive and have less risk of side effects. Therefore, a minimally invasive treatment, such as a topical cosmeceutical, is often the desired first line of defense.

However, many topical HA formulations contain HA molecules that are in fact too large to pass through the outer layer of the skin, and therefore are not very effective at treating signs of aging, other than by hydrating the surface layer of the skin. Studies have found that HA molecules need to have a molecular weight of less than 500 kDa in order to be able to effectively penetrate the skin barrier.

Other topical formulations containing active ingredients such as retinol and backuchiol are well known and are also marketed for their anti-aging benefits. Retinoids work by causing an inflammatory or stress response in skin cells which results in apoptosis. However, at doses above physiological levels retinol instead results in necrosis, as does bakuchiol. Essentially these active ingredients cause apoptotic and/or necrotic cell death which then stimulates cell renewal and turnover by increasing collagen and HA production. However, since these ingredients are essentially causing skin cells to die and the skin cells to be in a constant state of stress response, the long-term impacts of continued use of these types of harsh ingredients are unknown.

There is a growing demand for more effective, non-invasive, and more gentle anti-aging products containing active ingredients that are able to be delivered in sufficient concentrations to the intended target, where they can, if applicable, deliver a specific biochemical mechanism of action in the target cells or skin tissue.

There is also a growing demand for more effective products for preventing and treating the symptoms of GSM, many of which are linked to the depletion of HA during aging, including urinary incontinence, vaginal dryness, vaginal atrophy, irritation, itching, cracking, thinning, redness, inflammation and declining sexual function, sexual response and libido.

It is an object of the invention to provide pollen-based compositions and/or extracts and/or formulations or compositions comprising same, which will address the above-mentioned needs, or which will at least provide the public with a useful choice.

### SUMMARY OF THE INVENTION

In a first broad aspect the invention relates to a pollen-based composition, or an extract derived therefrom, wherein the composition or extract comprises at least glucose-6-phosphate, as a substrate or precursor for the production of hyaluronic acid (HA). Preferably, glucose-6-phosphate is present in the composition or extract in an amount of at least 0.05% by weight, and preferably in the range of about 0.05-5.0% by weight.

The pollen-based composition or extract of the invention preferably also comprises other small molecular weight bioactive molecules and/or compounds selected from the group comprising flavonoids, flavonols, flavones, alkaloids, aglycones, glycosides, lipids, fatty acids, amino acids, enzymes including hexokinases, glucokinases, proteases, peptidases, magnesium and other elements, anthranilic acid and isomers thereof, spermidine and isomers thereof, caffeoyl spermidine compounds, feruloyl spermidine compounds, and hydroxycinnamic acids.

The pollen-based composition or extract preferably comprises one or more spermidine isomers and/or caffeoyl spermidine compounds and/or feruoyl spermidine compounds, and/or compounds that are cinnamic acid conjugates of spermidine.

The pollen-based composition or extract preferably comprises one or more compounds selected from the group comprising tricaffeoyl-spermidine (N1,N5,N10-tricaffeoyl spermidine), triferuloyl-spermidine (N1,N5,N10-Tri-feruloyl spermidine), and dicaffeoyl-p-coumaroyl-spermidine.

The pollen-based composition or extract preferably comprises one or more compounds selected from the group comprising hydroxycinnamic acids. Preferably the hydroxycinnamic acids are selected from the group comprising dicaffeoylquinic acids and dicaffeoyl tartaric acid and isomers thereof.

The pollen-based composition or extract preferably comprises one or more flavonoids, flavonols and/or flavones. Preferably the flavonoids, flavonols and/or flavones are selected from the group comprising alkaloids, glycosides, and aglycones, including isorhamnetin, rhamnetin, tricetin, kaempferol.

The pollen-based composition or extract preferably also comprises one or more lipids. Preferably the lipids are selected from the group comprising fatty acids including myristic acid, palmitic acid, linoleic acid, stearic acid. Preferably the composition or extract also comprises lipids selected from the group comprising phospholipids, cholesterol and glycolipids. It is believed that the lipids and/or fatty acids act as carriers by encapsulating and protecting the other bioactive compounds in the composition or extract.

The terms “extract of the invention” or “extract derived from the composition” or similar as used herein are intended to refer to a concentrated version or concentrated extract of the pollen-based composition of the invention, where the extract comprises a more concentrated composition of the beneficial bioactive components identified above.

Concentration of the pollen-based composition of the invention can be carried out by methods known in the art such as centrifugation, membrane filtration, ultrafiltration and adsorption.

The invention also relates to formulations comprising the pollen-based composition or extract, with one or more carriers and/or excipients. Preferably the formulation is a topical formulation or an oral formulation such as a capsule or tablet. Preferably the topical formulation comprises between about 1-10% by weight of the pollen-based composition or extract. More preferably, the topical formulation comprises between about 1-4% by weight of the pollen-based composition or extract. An oral formulation of the invention may comprise a much higher amount of the pollen-based composition or extract, for example, at least 20% by weight, and more preferably between about 20-100% by weight.

In a further aspect, the invention relates to a method of stimulating the production of HA, comprising administering to an individual a therapeutically effective amount of a formulation comprising the pollen-based composition, or an extract derived from same. The stimulation of HA provides many health benefits, not only relating to the treatment of skin conditions and anti-aging, but also in relation to eye health, hair health, connective tissue and joint health, vaginal health, and other conditions where HA plays an important function.

In a further aspect, the invention relates to a method of treating or preventing vaginal atrophy, comprising administering to an individual in need of such treatment a therapeutically effective amount of a formulation comprising the pollen-based composition or an extract derived from same.

In a further aspect, the invention relates to a method of treating or preventing one or more of the symptoms of GSM, comprising administering to an individual in need of such treatment a therapeutically effective amount of a formulation comprising the pollen-based composition or an extract derived from same.

In still a further aspect, the invention relates to the use of a pollen-based composition or extract in the manufacture of a medicament for the prevention and/or treatment of one or more symptoms of Genitourinary Syndrome of Menopause (GSM), including urinary incontinence, vaginal dryness, vaginal atrophy, irritation, itching, cracking, thinning, redness, inflammation and declining libido, sexual function, sexual response and/or pleasure.

In still a further aspect, the invention relates to a method of producing a pollen-based composition comprising at least glucose-6-phosphate, the method comprising the steps of:
a. Combining one or more of raw bee pollen, raw honey and propolis with one or more of coconut water, coconut oil and/or coconut cream, to form a mixture;
b. Anaerobically incubating the mixture at a temperature of between about 25° C. to 65° C. for a period of between about 12 to 96 hours;
c. Freezing and/or freeze drying the resulting fermented composition.

In still a further aspect, the invention relates to a method of producing an extract derived from a pollen-based composition, said extract comprising at least glucose-6-phosphate, the method comprising the steps of:
a. Combining one or more of raw bee pollen, raw honey and propolis with one or more of coconut water, coconut oil and/or coconut cream, to form a mixture;
b. Anaerobically incubating the mixture at a temperature of between about 25° C. to 65° C. for a period of between about 12 to 96 hours;
c. Concentrating the resulting fermented composition;
d. Freezing and/or freeze drying the concentrated composition.

Preferably the concentration step is carried out by centrifugation, and collection of the resulting supernatant for freezing and/or freeze drying.

Preferably the composition is frozen for a period of up to 8 weeks, and then freeze dried, after which the freeze-dried composition is milled to a powder to produce a freeze-dried powdered composition that can be incorporated into various product formats.

In a further aspect, the invention relates to a method of treating or preventing a condition associated with a decline in hyaluronic acid production or degradation of hyaluronic acid, said method comprising administering to the subject a therapeutically effective amount of a formulation comprising the composition or extract as described herein.

In a further aspect, the invention relates to the use of a composition or extract as described herein, in the manufacture of a medicament for the treatment or prevention of a condition selected from the group comprising skin disorders, skin aging, Genitourinary Syndrome of Menopause (GSM) including urinary incontinence, vaginal dryness, vaginal atrophy, irritation, itching, cracking, thinning, redness, inflammation and declining libido, sexual function, sexual response and/or pleasure, dry eyes, connective tissue and joint disorders, and other conditions that may benefit from the stimulation or stabilization of hyaluronic acid (HA).

### BRIEF DESCRIPTION OF THE DRAWINGS

FIG. 1 is a flow diagram outlining the steps of manufacture of a composition or extract of the invention.

FIG. 2 is a photograph showing an extract of the invention after the centrifugation step.

FIG. 3 is an SDS-PAGE image protein analysis of samples taken at various time intervals during the incubation step.

FIG. 4 is an SDS-PAGE image of the samples of FIG. 3 indicating visible protein bands and their corresponding molecular weights.

FIG. 5 depicts a RP-UHPLC and negative ion electrospray ionization (ESI) analysis of a pollen-based composition prepared according to Example 1.

FIG. 6 is a comparative graph showing the efficacy of a pollen-based composition of the invention prepared by Example 1 at stimulating the production of HA in human keratinocytes.

FIG. 7 (a) to (c) are photographs showing growth of skin cells achieved during efficacy study measuring the level of cellular proliferation and differentiation of a composition and extract of the invention.

FIGS. 8 (a) to (f) are UV chromatograms of the original fractions 5-10 of the pollen-based composition identified by the RP-UHPLC analysis of FIG. 5.

FIGS. 9 and 10 (a) to (d) are UV chromatograms of the original fractions 25-30 of the pollen-based composition identified by the RP-UHPLC analysis of FIG. 5.

FIG. 11 is a PrestoBlue analysis of keratinocytes exposed to extract of the invention (MYR) and subfractions of the extract, in comparison to controls.

FIG. 12 and FIG. 13 show some further UV chromatography analysis conducted in respect of the more active subfractions of FIG. 11.

FIGS. 14-17 are graphs showing the results of a trial of a lip balm formulation comprising an extract of the invention.

FIGS. 18-21 are graphs showing the results of a trial of a vaginal cream formulation comprising an extract of the invention.

### DETAILED DESCRIPTION

A detailed description of one or more embodiments of the invention is provided below along with accompanying tables and figures that illustrate the principles of the invention. As such this detailed description illustrates the invention by way of example and not by way of limitation. The description will clearly enable one skilled in the art to make and use the invention, and describes several embodiments, adaptations, variations and alternatives and uses of the invention, including what the inventor presently believes is the best mode for carrying out the invention. It is to be clearly understood that routine variations and adaptations can be made to the invention as described, and such variations and adaptations squarely fall within the spirit and scope of the invention.

Raw bee pollen contains many beneficial vitamins, minerals, nutrients, proteins, amino acids, lipids, fatty acids, enzymes, antioxidants, carotenoids and bioflavonoids. The exact composition of bee pollen varies depending on the plant source and the season in which the pollen is collected. It is widely reported that raw bee pollen may have numerous health benefits including antibiotic, antimicrobial, anti-inflammatory and antioxidant benefits.

Raw bee pollen is naturally in granulated form, whereby the pollen grains have a hard shell known as the sporopollenin which is very resistant to chemical degradation. In addition, on the surface of the pollen are proteins which cause known allergies. It is therefore desirable, but difficult, to “open” the pollen grains to access or release the beneficial components inside.

In previous research, the inventor devised a unique method of treating pollen grains to stimulate natural germination of the pollen grains, to naturally and gently release their contents. This “biotic” method of stimulating pollen grains is described in WO 2015/028892, which is hereby incorporated by reference. Also described is a two-stage process for producing a pollen-based extract material, including opening and/or germinating the pollen grains using the biotic method, and then reacting the germinated pollen grains with one or more beehive components, such as, for example, beeswax, honey, propolis, and other components such as glycerin, and enzyme ingredients, to obtain an extract and compositions comprising the extract that were beneficial for treating skin disorders such as eczema, psoriasis and acne.

The inventor has carried out further research and development since producing the extracts and compositions disclosed in WO 2015/028892. In particular, research was focused on producing new extracts and compositions with increased bioactivity, and less non-essential or non-beneficial components such as large molecules that cannot be fully absorbed by the skin, and large proteins which are known to be potential allergens. The further research and development focused on refining the ingredients and the process of producing pollen-based compositions and extracts and identifying and concentrating the most beneficial components in order to obtain improved pollen-based extracts, and compositions comprising same.

As a result, the inventor has devised an improved process of preparing a fermented pollen-based composition or extract, which has been found to exhibit surprising properties, and additional advantages. In particular, the inventor has surprisingly discovered that pollen-based extracts and compositions can be produced that are able to stimulate the production of hyaluronic acid, amongst other things, by a unique mechanism of action, that is different and much gentler than the harsh action of anti-aging ingredients such as retinol and backuchiol.

The inventor has found that the improved extract and compositions of the invention stimulate hyaluronic acid production in keratinocytes, and induce cellular proliferation and differentiation. In addition, the extract and compositions of the invention are antimicrobial to acne propionic bacterium, Staphylococcus aureus and Candida (thrush). These factors are central to GSM and the associated symptoms of vaginal atrophy, vaginal dryness, burning, itching, irritation, redness, inflammation, loss of plumpness, microbiome imbalance (leading to an increased risk of UTI infection and thrush), and declining libido and sexual function. Accordingly, the inventor has found that the extract and compositions of the invention are extremely useful in the prevention and treatment of GSM and the symptoms associated therewith.

### Methods of Manufacture

The pollen-based composition of the invention is produced using two main groups of ingredients, the “beehive ingredients” and the “coconut ingredients”. The beehive ingredients include raw bee pollen, raw honey and propolis. The beehive ingredients, can be sourced from any beehive, but are preferably sourced from beehives in the Southern Alps region of New Zealand where there are beech forests. Preferably, the raw bee pollen is cold shocked before use because cold shocking turns on the apoptosis reversal or protection response in plant cells, which appears to enhance the resulting beneficial bioactive components of the resulting composition. The honey can be any type of pure raw honey or raw honey blend. It is important that the honey is not pasteurized honey. Some examples of suitable honey include honeydew honey, manuka honey, rewarewa honey, clover honey, beech honey, and pohutukawa honey. Raw propolis is preferably used, or alternatively, propolis can be prepared for use as a water or isopropyl alcohol extraction by combining 25 g propolis with 50 mL isopropanol or water in a dark sealed glass vessel, with gentle rotation for 28 days at 28° C. The raw propolis can either be added into the mixture at the start of the process, or alternatively the isopropanol form of propolis can be added at the end of the incubation period.

The coconut ingredients include one or more of coconut water, coconut oil and/or coconut cream. The coconut ingredients can be obtained either by extracting them from fresh coconuts, or alternatively, commercially available coconut water, coconut cream and coconut oil can be used. The coconut water facilitates the natural and gentle opening or germination of the pollen grains which preserves the beneficial bioactive components therein. The coconut oil is used to facilitate anerobic fermentation (but is not required if an airtight vessel is used) and also to provide a source of fatty acids, which are thought to facilitate the delivery of the bioactive components in the extract, such as flavonoids, to the skin.

The pollen-based composition of the invention, and extracts derived from same, are preferably dried powdered compositions, and more preferably freeze-dried powdered compositions.

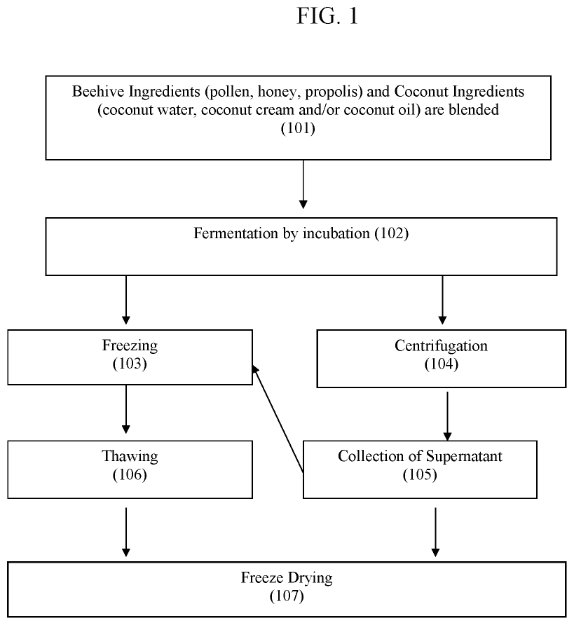

Referring to FIG. 1, a freeze-dried pollen-based composition can be prepared using the following steps. Firstly, the selected beehive ingredients are combined with one or more of the coconut ingredients and blended (101). The mixture is anaerobically incubated (102) at a temperature of between about 25° C. to 65° C. for a period of between about 12 to 96 hours. Incubation at a temperature of about 37° C. is preferred as it is an optimal temperature for enzyme activity and speeds up the reaction so the reaction may be completed in about 48 hours. Alternatively, the mixture could be anaerobically incubated at a temperature of about 25° C. for a longer period. It is believed that a longer incubation period is linked to an increased production of isomers of the key components of the extract. However, it is important not to allow the incubation period to go on too long, to avoid any production of ethanol and degradation of isomers. If a shorter incubation period of about 12 hours is employed then the incubation temperature should be higher, for example, up to about 65° C. which will significantly speed up the reaction.

After the incubation period, propolis can be added, if it was not added at the start of the process. Then the resulting composition can either be frozen immediately (103), by placing in a freezer for a period of up to eight weeks.

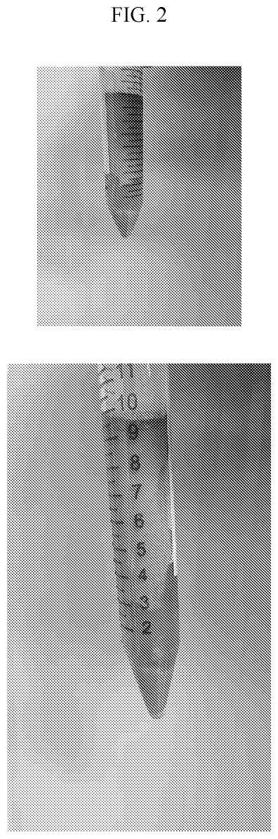

Alternatively, and preferably, the resulting composition is centrifuged (104) to separate the components of the composition and produce a more concentrated extract. The centrifugation step is carried out using methods known in the art. It is important that the key components of the composition are retained during the centrifugation process. The inventor used a process of bioactivity guided fractionation to ensure that the key beneficial bioactive components of the composition (described above) were retained in the supernatant phase. In the examples described below, the rpm was set at 9600, the flow was set at 400 liters per hour and the discharge of the heavy phase was set at 200 seconds. This was based on a centrifuge made by Alfa Laval, however the centrifuge parameters could obviously be varied as necessary to suit different equipment, and as needed to suit different applications. The supernatant phase is then collected (105) and may or may not be frozen (103). The collected supernatant has a gel-like texture and is a rich yellow/orange colour as shown in FIG. 2. Preferably, the collected composition is initially frozen in a blast freezer for up to 48 hours. Thereafter it is preferably frozen at −18° C. for up to eight weeks to reduce the microbial load. The longer the composition is frozen for, the lower the microbial count is. If the composition is not frozen immediately, the microbial count may be high.

Alternatively, the collected supernatant could be dried, preferably by freeze-drying, without a preceding freezing step. The freeze-drying parameters could be varied based on the knowledge of persons skilled in the art and the desired application of the composition or extract.

If the composition or extract derived therefrom has been frozen, the composition or extract is then thawed (106) and then dried, preferably by freeze-drying (107) by methods known in the art. The resulting freeze-dried composition may be milled to a powder or further processed by methods known in the art. The composition or extract of the invention can then be incorporated in various product formats including topical compositions, or oral compositions such as capsules or tablets as discussed further below.

The composition and extract of the invention has a very low microbial count (<10 cfu) if the product is frozen and then freeze dried in accordance with the process outlined above.

However, if the composition or extract is to be used in a pharmaceutical grade product, where it is necessary to have a lower microbial count, further sterilization steps could be carried out as known in the art.

It is envisaged that the composition or extract of the invention could undergo further processing steps by methods known in the art, in order to produce further purified and/or more concentrated extracts of the invention, in which specific bioactive components that are responsible for certain beneficial effects and/or mechanisms of action are isolated and concentrated. These purified or concentrated extracts could then be included in various formulations or compositions to deliver specific products with specific mechanisms of action and/or to target specific needs.

### Protein Content of Composition

The inventor surprisingly found that it was not necessary to use any additional enzymes or enzyme-containing ingredients in the manufacture of the composition of the invention. The addition of enzymes or enzyme-containing ingredients, such as pineapple extract, was found to inhibit the digestion of protein in the composition rather than enhance it. Instead, it was surprisingly found that if the incubation period was extended to between 48 to 72 hours, there were no proteins remaining in the composition. All of the protein in the composition was completely broken down into pure amino acids.

During the incubation period, six liquid samples were taken at various time points, so they could be analysed for protein/peptide content. The samples were taken at T=0; T=24 hours; T=48 hours; T=72 hours; T=96 hours; and T=196 hours. The samples were analysed using sodium dodecyl-sulfate polyacrylamide gel electrophoresis (SDS-PAGE).

Each of the six liquid samples were diluted with water at 10-fold first, then treated with a sample buffer (Bolt™ LDS, Invitrogen™) at a 3:1 ratio before being heated at 70° C. for 10 minutes. Treated samples were then loaded (15 μL each) into the lanes of two Bis-Tris gels (NuPAGE™ 4-12%, Invitrogen™). The gels were visualised after Coomassie Blue staining, and an image was taken and analysed on a Gel doc system (Essential V2, UVITEC, Cambridge).

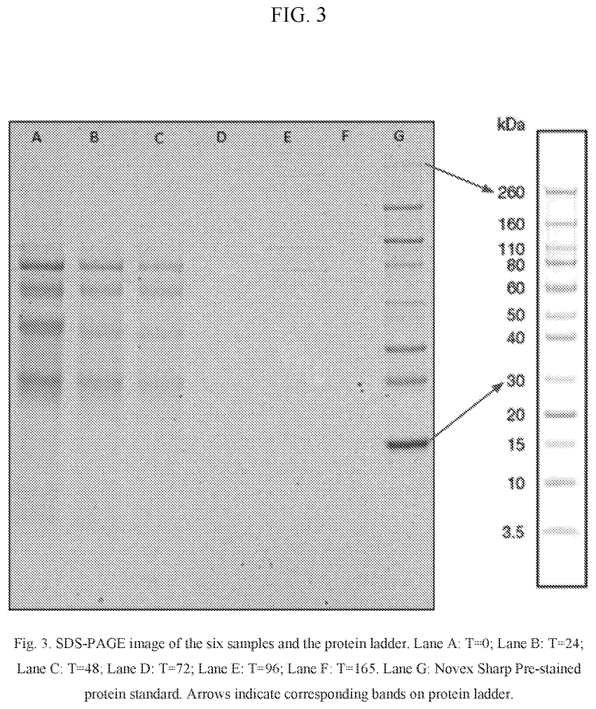

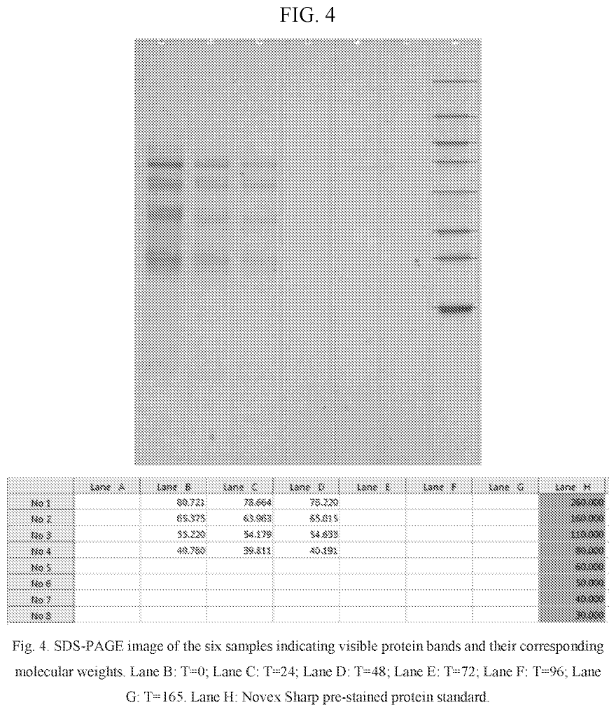

The image of the PAGE was analysed and the molecular weights of the major protein bands in each sample were recorded. FIG. 3 shows an image of the protein bands from the samples. FIG. 4 provides the molecular weight of each visible protein band in the samples shown in FIG. 3. These Figures show that there was a steady drop in protein concentration from T=0 to T=48 hours, after which the bands have entirely disappeared at T=72 hours, with no obvious protein bands. This confirms that complete digestion of protein occurred during the incubation period between 48 to 72 hours.

The inventor believes that the germination and gentle fermentation process releases enzymes including proteases, peptidases, hexokinases and glucokinases from the beehive ingredients, namely the pollen grains and the honey, including from Bacillus subtilis present in the beehive ingredients, and these enzymes are responsible for digesting the protein components and for producing glucose-6-phosphate which is a precursor to HA production.

The methods described above may be further understood in connection with the following Examples.

### Example 1—Preparation of Composition

Coconut water and raw bee pollen was mixed with coconut oil, honeydew and raw propolis. All ingredients were incubated at 37° C. for 72 hours. The resulting composition was frozen for a period of up to 8 weeks to stabilize the composition. After freezing, the composition was thawed and then freeze dried and milled to a powder.

### Example 2—Preparation of Concentrated Extract

Coconut water and raw bee pollen was mixed with coconut oil, honeydew and raw propolis. All ingredients were incubated at 37° C. for 72 hours. The resulting composition was centrifuged. The centrifuge used was made by Alfa Laval and the rpm was set at 9600. The flow was set at 400 liters per hour and the discharge of the heavy phase was set at 200 seconds. The supernatant was then collected and frozen for a period of up to 8 weeks to stabilize the composition. After freezing, the composition was thawed and then freeze dried and milled to a powder.

### Example 3—Preparation of Concentrated Extract

Coconut water and raw bee pollen was mixed with coconut oil, honeydew and raw propolis. All ingredients were incubated at room temperature (25° C.) for up to 96 hours. The resulting composition was centrifuged. The centrifuge used was made by Alfa Laval and the rpm was set at 9600. The flow was set at 400 liters per hour and the discharge of the heavy phase was set at 200 seconds. The supernatant was then collected and frozen for a period of 8 weeks to stabilize the composition. After freezing, the composition was thawed and then freeze dried and milled to a powder.

### Example 4—Preparation of Concentrated Extract

Coconut water and raw bee pollen was mixed with coconut oil, honeydew and raw propolis. All ingredients were incubated at about 65° C. for 12 hours. The resulting composition was centrifuged. The centrifuge used was made by Alfa Laval and the rpm was set at 9600. The flow was set at 400 liters per hour and the discharge of the heavy phase was set at 200 seconds. The supernatant was then collected and frozen for a period of up to 8 weeks to stabilize the composition. After freezing, the composition was thawed and then freeze dried and milled to a powder. The higher incubation temperature allowed the reaction to be completed in a shorter time period, but this did not impact the protein content of the extract as the extract was still found to contain no protein content.

### Antimicrobial Benefits of Composition

A composition of the invention prepared in accordance with Example 1 was tested for antimicrobial activity by growing the test organisms Propionibacterium acnes and Staphylococcus aureus on an agar plate and challenging the test organisms with the composition. The results are shown in the tables below.

| | ATCC | Organism Level Recovered (cfu/g) | | | | | |
|---|---|---|---|---|---|---|---|
| Organism | No. | Inoculum | Day 0 | Day 2 | Day | Day 14 | Day 28 |
| Propionibacterium | 6919 | 1.6 × 10⁷ | 1.9 × 10⁵ | <10 | <10 | <10 | <10 |
| acnes | | | | | | | |
| Staphylococcus | 6538 | 8.3 × 10⁶ | 1.0 × 10⁶ | 2.3 × 10² | <10 | <10 | <10 |
| aureus | | | | | | | |
| Propionibacterium | 6919 | 7.2 | 5.3 | <1 | <1 | <1 | <1 |
| acnes | | | | | | | |
| Staphylococcus | 6538 | 6.9 | 6.0 | 2.4 | <1 | <1 | <1 |
| aureus | | | | | | | |

| | | Log Difference | | | | | |
|---|---|---|---|---|---|---|---|
| | | Between | Between | Between | Between | Between | Between |
| | ATCC | Inoculum | Inoculum | Inoculum | Inoculum | Inoculum | Day 14 and |
| Organism | No. | and Day D | and Day 2 | and Day 7 | and Day 14 | and Day 28 | Day 28 |
| Propionibacterium | 6919 | 2 | >7 | >7 | >7 | >7 | N [symbol] |
| acnes | | | | | | | |
| Staphylococcus | 6538 | 1 | 5 | >6 | >6 | >6 | N [symbol] |
| aureus | | | | | | | |

NB. Rounding of log differences from 0.5 onward to next whole number has been applied. [symbol]
indicates data missing or illegible when filed

### Chemical Analysis of Composition and Extract

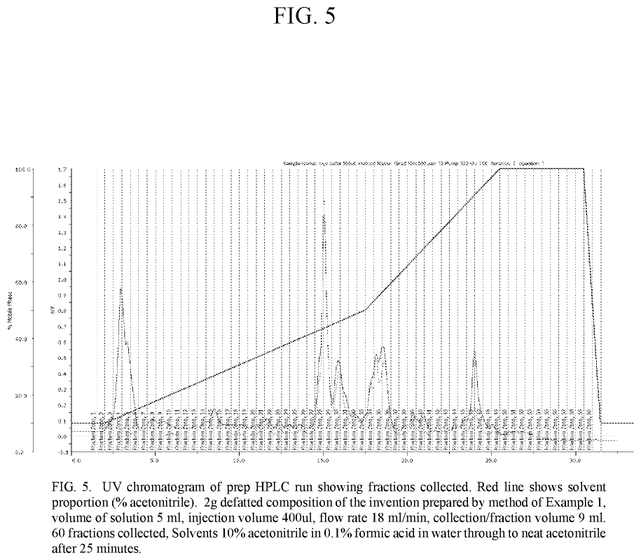

FIG. 5 is an HPLC analysis of the composition of the invention prepared in accordance with Example 1.

The same HPLC profile was observed in the analysis of concentrated extracts prepared by the methods described in Examples 2-4.

Following the HPLC analysis, the following subfractions (27 in total) of the 60 fractions, were collected for further analysis using bioassay-guided fractionation, to identify the key bioactive components in the extract:

TABLE 1
Fractions collected for bioassay-guided fractionation-
based on the UV chromatography profile of FIG. 5

| | Original fraction |
|---|---|
| Combined Fraction No. | numbers |
| 1 | 1, 2 |
| 2 | 3 |
| 3 | 4 |
| 4 | 5 |
| 5 | 6, 7 |
| 6 | 8, 9, 10 |
| 7 | 11, 12, 13 |
| 8 | 14, 15, 16 |
| 9 | 17, 18 |
| 10 | 19, 20 |
| 11 | 21, 22 |
| 12 | 23, 24 |
| 13 | 25, 26 |
| 14 | 27, 28 |
| 15 | 29, 30 |
| 16 | 31, 32 |
| 17 | 33, 34 |
| 18 | 35, 36 |
| 19 | 37, 38 |
| 20 | 39, 40 |
| 21 | 41, 42 |
| 22 | 43, 44 |
| 23 | 45, 46, 47 |
| 24 | 48, 49 |
| 25 | 50, 51, 52 |
| 26 | 53, 54, 55 |
| 27 | 56, 57, 58 |

### Bioassay-Guided Fractionation

A series of bioassays were carried out to investigate the efficacy of the composition and extracts of the invention in terms of their effects on HA production in skin cells and on their proliferative effects on skin cells.

### HA Bioassay

Keratinocytes (skin cells) were grown in a single layer in tissue culture overnight and then treated for a period of four hours with the following test substances:
a. a composition of the invention prepared in accordance with Example 1 (whole composition),
b. a concentrated extract of the invention prepared in accordance with Example 2 (extract),
c. each of the 27 subfractions identified in Table 1 above,
d. retinol (as a positive control) and
e. blank (untreated) (culture media only).

Cells were then harvested, lysed and then analysed using the Perkin Elmer Elisa Assay (also known as AlphaLISA HA assay) to measure production of HA within the skin cells after four hours. The skin cells used were S140 Keratinocytes P2 sourced from a single human donor. The retinol was Vitamin A Palmitate 1 million IU/Retinyl Palmitate/Retinol sourced from Pure Nature. Materials required included 10 μl of cell culture supernatant; 1×96 well plate; 500 assay points—48 well required for standard each time the assay is done; triplicate for each sample.

### Method Steps:

a. Dissolve the test substances in DMSO. Leave to dissolve overnight at room temperature and then move to the fridge until ready for use;
b. Seed 5000 keratinocytes per well in 200 μl of Kelch's medium into all required wells;
c. Incubate at 37° C./5% CO₂ for 24 hours;
d. Make a serial dilution of the test substances in DMSO so that all samples receive the same amount of DMSO no matter the concentration of the test substances;
e. Remove all media and replace with 200 μl of Kelch's medium containing the appropriate concentration of each of the test substances, including DMSO and retinol (diluted in DMSO), laid out in a 96 well plate;
f. Incubate at 37° C./5% CO₂ for 4 hours;
g. Image wells after 4 hours to assess visible effects of treatment;
h. Remove all cell culture supernatant and store at −20° C. until ready to carry out HA assay;
i. Carry out HA assay on the test substances.

### Results:

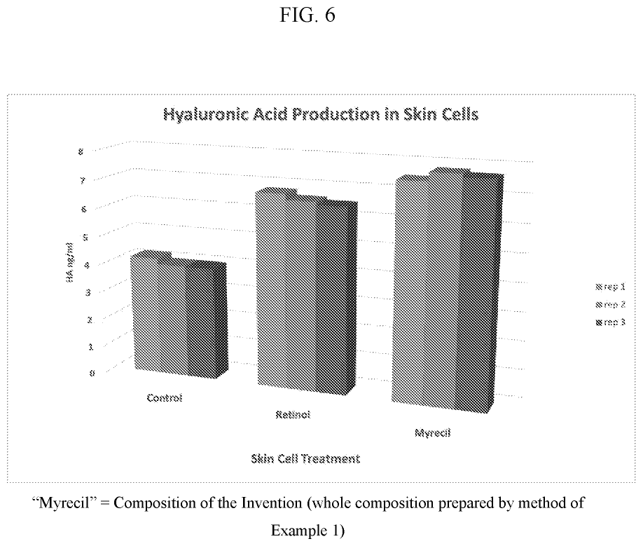

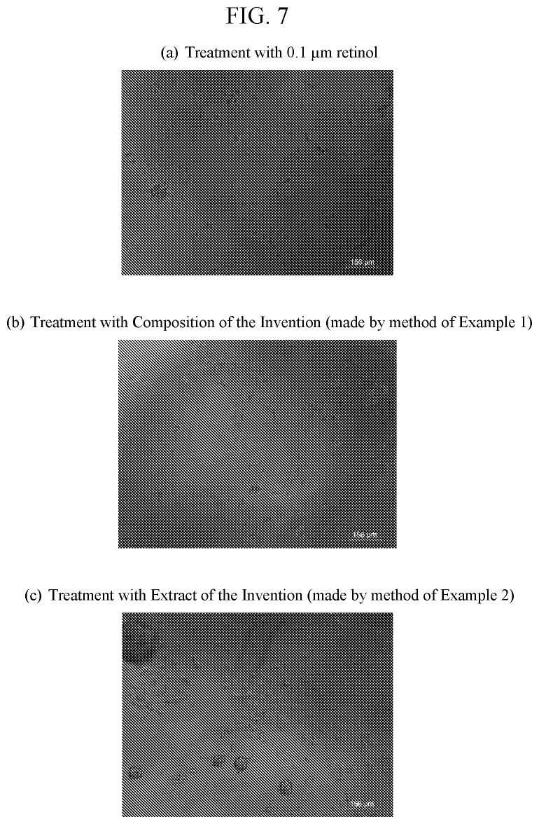

FIG. 6 is a graph showing the results of the HA assay of the whole composition of the invention. It was surprisingly found that when treated with the composition of the invention, the skin cells produced more HA than the skin cells of the control sample, and more HA than skin cells treated with retinol, which is widely considered by experts and dermatologists to be an effective active product which works at a deep cellular level to reduce cellular aging by promoting production of collagen, elastin, sebum and HA. Furthermore, the results show that the composition of the invention promoted the growth of fibroblasts and scaffolds, as shown in the photograph in FIG. 7 (b).

The concentrated extract of the invention prepared by the method of Example 2 exhibited an even higher bioactivity than the composition of the invention, as can be seen from the photograph in FIG. 7 (c), which shows extraordinary results in the growth of fibroblasts and scaffolds.

In terms of the subfractions tested, subfractions 4, 5, 6, 13, 14 and 15 exhibited the most efficacious results out of the subfractions, and these fractions were selected for further bioassay-guided fractionation to identify the important bioactive components in the composition and extract of the invention that are most likely contributing to the stimulation of HA production.

The HA bioassay revealed that the activity of the composition and extract was centered around two areas in the chromatogram. To lessen confusion, the tested combined fractions in Table 1 are referred to as test fractions while the collected fractions are referred to as original fractions.

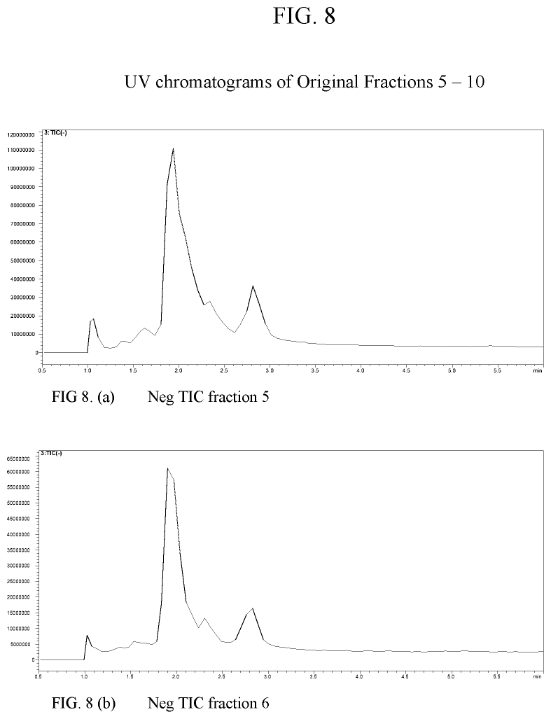

These areas were:
Area 1: Activity in test fractions 4 (+), 5 (++) and 6 (+++). (Original fractions 5-10)
Area 2: Activity in test fractions 13 (+), 14 (++) and 15 (+++). (Original fractions 25-30)
FIGS. 8-10 are UV chromatograms of each of the original fractions 5-10 and 25-30.
   Area 1—Shown in FIG. 8 (a)-(f)

This area or the chromatogram is relatively polar, eluting just after the main initial peak. LCMS analysis of the original fractions show fractions 5 and 6 are similar with little sign of MS peaks past RT 3 mins. Fractions 7-10 show reducing levels of these early peaks the area after 3 minutes showing small peaks in the TIC. As fraction 9 and 10 are the most active (test fraction 6) it is likely the activity is associated with the peaks eluting after 3 minutes. The MS data in this area of the chromatogram is complex. There is a negative ion peak at 326 amu which could be related to the activity.

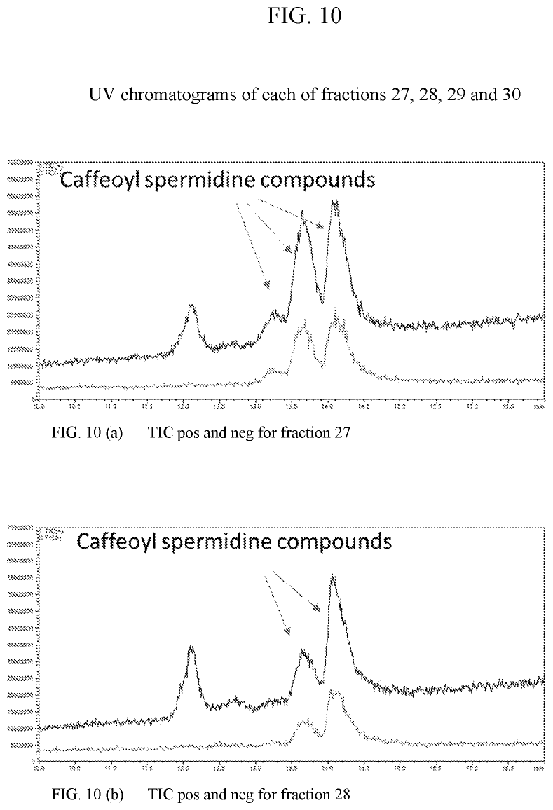

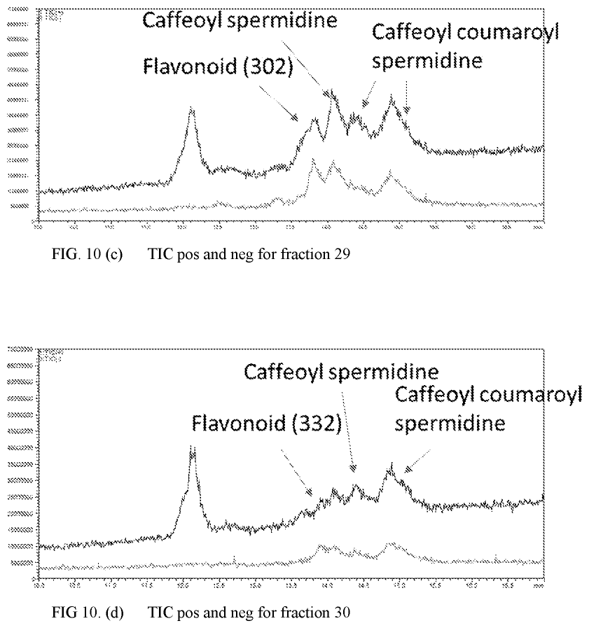

Area 2—Shown in FIG. 10 (a)-(d)

This area is around the 15 min elution time in the preparative HPLC run. The least active fraction (test 13) is the original fractions 25 and 26 which is just before the major UV peak, the next most active is original fractions 27 and 28 which are collected from the main UV peak while the most active fractions (29 and 30) are just at the tail end of the major UV peak. These fractions are more amenable to LCMS analysis. The two fractions 27 and 28 are mainly comprised of the caffeoyl spermidine compounds. These compounds appear as three peaks (isomeric) with the first isomer at a much lower level. The mass spectra of these compounds show clear strong ions at 630 (neg) and 632 (pos).

The most active fractions 29 and 30 show more complexity. These two fractions are also similar to each other. There is evidence of some of the caffeoyl spermidine compounds (neg ms 630) and a later peak with neg MS 614. This could be another spermidine compound with a mix of caffeic and coumaroyl acylation (accounting for one less oxygen). In fraction 29 the pattern and MS data suggests two isomers of this spermidine compound, while the later peak is more dominant in fraction 30.

Fractions 29 and 30 also show the presence of several other peaks of interest. In 29 there is a MS peak for MW 300 (301 neg, 303 pos), while in 30 there are two others with MW 332 (331 neg, 333 pos) and 380 (379 neg, 381 pos). The UV data suggest these peaks may be due to flavonoid type compounds although the spectra are somewhat overlapped with the spermidine compounds. Note the MW for these compounds is indicative of no glycosylation of these compounds.

Other compounds of interest in fractions 25-30 include but are not limited to anthranilic acid, dicaffeoylquinic acid, and dicaffeoyl tartaric acid.

Since the major activity is associated with fractions 29 and 30 it is likely that the minor flavonoids and the isomeric dicaffeoylquinic acids and dicaffeoyl tartaric acid, rather than the spermidine compounds, are the more important bioactive compounds contributing to the beneficial effects of the composition and extracts derived therefrom.

However, it is important to note that original fractions 1-4 would have contained very small water-soluble molecules and compounds that would have been lost or eluted in the early phase of the chromatogram and therefore not included in the bioassay-guided fractionation. The inventor believes that these fractions contain hexokinase, glucokinase, glucose-6-phosphate, and other small water-soluble molecules such as magnesium, which are very important contributors to the bioactivity of the composition and extract of the invention, as they provide the building blocks for HA production. These small water-soluble molecules and compounds are well retained in the composition and concentrated extracts of the invention when prepared by the methods described above.

### Keratinocyte Proliferation Bioassay

Further analysis using bioassay-guided fractionation was carried out in respect of the concentrated extract to determine which compounds in the extract were likely responsible for the cell growth and proliferation which was observed in the HA bioassay.

The concentrated extract was fractionated based on polarity to produce 27 further subfractions labelled as follows: 8-8, 8-9, 8-10, 8-11, 8-12, 8-13, 8-14, 8-15, 8-16, 9-27, C1, C2, C3, C4, C5, C6, C7, C8, L1, L2, L3, LP23, LP25, LP30, LP36, LP37, LP38.

The concentrated extract (labelled MYR), together with each of the subfractions were then tested for efficacy by treating keratinocytes, and assessing any changes in proliferation and differentiation, and/or changes in morphology of the keratinocytes. These are characteristics that compounds such as retinol and retinal are known to induce, therefore, the keratinocytes were also treated with these compounds as a positive control.

### Preparation of Samples:

The concentrated extract of the invention was prepared for testing by making a 100 mg/ml stock concentration, dissolving the extract then transferring the supernatant without sediment to a fresh tube. The subfractions were prepared by adding 100 μl of DMSO to each tube supplied. The assumption was that each tube contains 55 mg of material. Original stock concentration is therefore 550 mg/ml. The subfractions were then diluted to 100 mg/ml working solutions in DMSO.

### Method:

1. Seed 5000 keratinocytes per well in 200 μl of Kelch's+SB+KGF medium into all required wells of two 96 well plates (0.75M in 30 ml of media or 0.5M in 20 ml of media).
2. Add DF10 media border around the plate.
3. Incubate at 37° C./5% CO₂ overnight.
4. Add 1 μl of 100 mg/ml working stock of all treatments to 249 μl of Kelch's+SB+KGF medium for cells treatment for final concentration of 400 μg/ml. Make up treatments in a 96 well flat bottom plate so the medias can be imaged after 24 hours.
5. Add 1 μl of DMSO to 249 μl of Kelch's+SB+KGF medium for cells treatment for final concentration of 400 μg/ml.
6. Add 1 μl of CM5 to 249 μl of Kelch's+SB+KGF medium for cells treatment for final concentration of 1.5 μg/ml.
7. 1 μl 0.1 mM retinol was added to 999 μl of Kelch's+SB+KGF medium for cells treatment for final concentration of 0.1 μM/ml. 250 μl was added to the wells for this treatment condition.
8. Add DF10 border to media plate.
9. Leave extract and subfractions in Kelch's medium at 37° C. for 24 hours to make sure extract doesn't aggregate in the cell culture medium. Check these for clumps and do not use if there are clumps.
10. Add 1 μl of 13 mg/mL retinal to 249 μl Kelch's+SB+KGF medium for cells treatment for final concentration of 52 μg/ml.
11. Add 1 μl of 13 mg/mL glycerol vehicle control to 249 μl Kelch's+SB+KGF for cells treatment for final concentration of 52 μg/ml.
12. Remove all media from keratinocytes and replace with 200 μl of Kelch's medium containing each treatment as laid out in the 96 well plate. Use a multichannel at this point.
13. Add DF10 media border around the plate.
14. Incubate at 37° C./5% CO₂.
15. Image wells after 24 hours to assess visible effects of treatment.

### PrestoBlue Assay Method:

1. Remove all media.
2. Add 150 μl of PrestoBlue and Kelch's+KFG+SB media mix at a 1:9 ratio to each well.
3. Incubate at 37° C./5% CO₂.
4. After 1.5-2 hours read the plate on the SpectraMax ID3.

The above experiments were repeated eight times with all samples in triplicate.

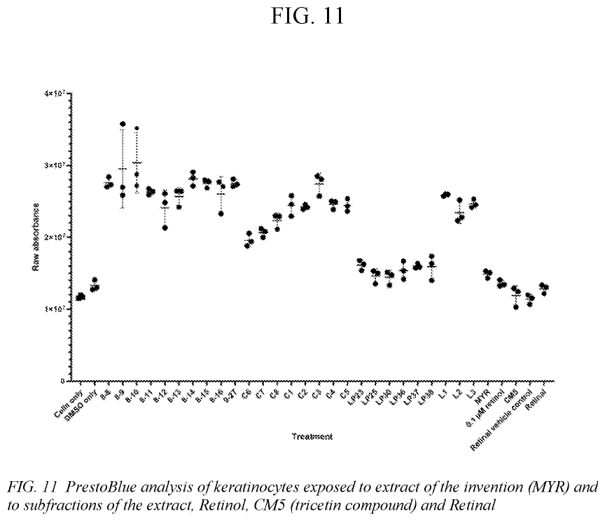

Statistical analysis and data were plotted in GraphPad Prism 10.0. FIG. 11 is a graph showing a summary of the results of the bioassay. All data in FIG. 11 is based on individual wells subtracted from the average of the background PrestoBlue signal with no cells.

### Results:

As can be seen in FIG. 11, the concentrated extract of the invention (labelled MYR) exhibited higher keratinocyte proliferation and differentiation than the positive controls, retinol and retinal.

Some of the subfractions showed significant increases in keratinocyte proliferation and differentiation, namely subfractions 8-8 to 8-16, 9-27, C3, C4, C5, L1, L2, L3, when compared to DMSO only control, and when compared to the positive controls.

### Analysis of Active Fractions

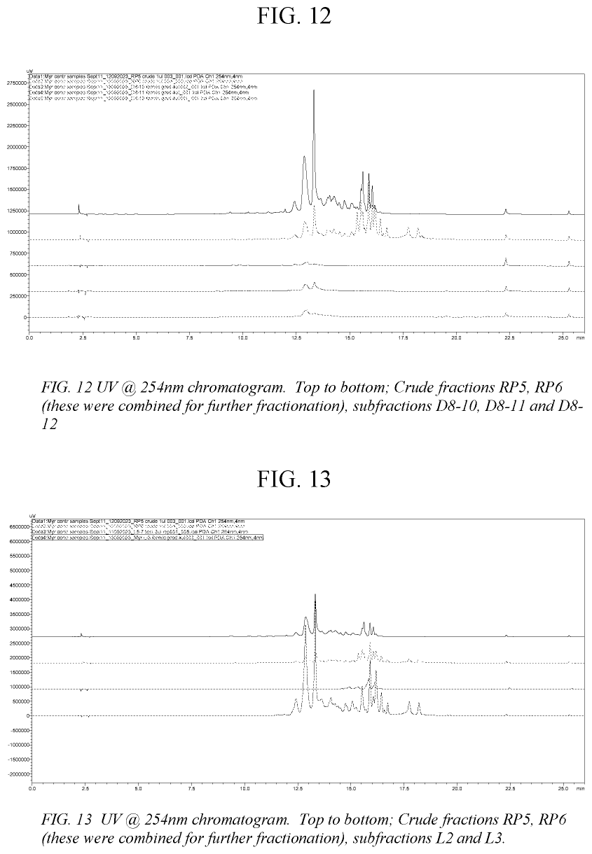

Some further UV chromatography analysis was conducted in respect of some of the most active subfractions, the results of which are shown in FIG. 12 and FIG. 13.

In relation to FIG. 12, the chromatogram shows that in Fraction D8-11, two UV peaks and two corresponding main mass spectrometer peaks are observed. The earlier peak is broader and is likely to represent more than one compound. The dominant mass for both peaks is 630 in negative mode and 632 in positive mode. This is almost certainly tricaffeoyl-spermidine (MW 631). A comparison of D8-11 with D8-12 and D8-10 suggests the activity is linked to the later of the two peaks. The later tri-caffeoyl spermidine peak is sharp which suggests this may represent the trans isomer of the compound. The earlier peak(s) could then be a mix of cis and trans isomers.

Underneath the earlier peak there is evidence for the presence of two flavonoid compounds with negative MS peaks at 310 and 331. There is also a suggestion in the UV spectra for this peak of the presence of other compounds, not just caffeoyl compounds, which are most likely to be further flavonoid compounds.

In relation to FIG. 13, the chromatogram shows that the activity associated with the L2 fraction is not associated with tri-caffeoyl spermidine compounds, since fraction L3 is the fraction that is rich in these compounds. Further analysis of the L2 fraction indicates the presence of a pair of peaks with pos. MS 674, which after ms/ms analysis indicates this may be tri-feruloyl spermidine (that is, the o-methyl analogues of the two peaks in fraction D8-11). In the L fractions, the minor flavonoids have been separated from the more prominent flavonoids, namely the spermidines. Since the activity in the L fractions is high, this further indicates that it is not just the spermidine compounds that are responsible for stimulating HA production and cellular proliferation and differentiation in skin cells. Other compounds present in the L fractions include dicaffeoylquinic acids, and tricetin.

The bioassay-guided fractionation analysis of the composition and concentrated extract of the invention, clearly show that it is more than a single compound that is responsible for the efficacy observed, namely the increase in production of HA, and increased proliferation and differentiation in skin cells.

Surprisingly, it was found that the most beneficial components of the composition and extract of the invention are not proteins, peptides or peptolipids.

The inventor believes that the following classes of compounds are present in the compositions and extracts of the invention, and are the main contributors to the beneficial bioactivity of the compositions and extracts, and the beneficial effects observed with use of formulations comprising the compositions and extracts as an active ingredient:
Spermidine isomers, caffeoyl spermidine compounds, feruoyl spermidine compounds, and compounds that are cinnamic acid type conjugates of spermidine, including tricaffeoyl-spermidine (N1,N5, N10-tricaffeoyl spermidine), triferuloyl-spermidine (N1,N5,N10-Tri-feruloyl spermidine), and dicaffeoyl-p-coumaroyl-spermidine;
Hydroxycinnamic acids including dicaffeoylquinic acids and dicaffeoyl tartaric acid and isomers thereof;
Flavonoids (flavonols and/or flavones) including alkaloids, glycosides, and aglycones such as isorhamnetin, rhamnetin, tricetin, kaempferol;
Small water-soluble molecules and compounds including glucose-6-phosphate, hexokinase, glucokinase, proteases, peptidases, amino acids, magnesium and other elements, anthranilic acid and isomers thereof;
Lipids, namely fatty acids including myristic acid, palmitic acid, linoleic acid, stearic acid, and phospholipids, cholesterol and glycolipids.

The inventor believes that the lipids and/or fatty acids present in the extract act as carriers by encapsulating and protecting the other bioactive compounds in the extract.

Since the composition was manufactured without the addition of any enzyme-containing components, but the composition still exhibited enzymatic activity in terms of eliminating the protein content, an analysis of the enzymes present was carried out. This revealed that as well as proteases and peptidases responsible for digesting the protein over the 72 hours as shown in the protein gels, hexokinases and glucokinases were also identified. These enzymes are known to be present in pollen, honeybee saliva and Bacillus subtillus.

Hyaluronic acid is produced by the phosphorylation of glucose by hexokinase to produce the main precursor of HA, namely, glucose-6-phosphate. Accordingly, the inventor believes that the induction of germination of the pollen by the coconut water releases hexokinases that then phosphorylate glucose to glucose 6 phosphate. From here, the HA synthesis pathway can be divided into two distinct pathways that synthesise the two building blocks of HA, namely glucuronic acid and N-acetylglucosamine. The inventor believes that the other components present in the composition or extract of the invention, as identified above, including the flavonoids, hydroxycinnamic acids and spermidine compounds, activate one or both of these pathways to induce hyaluronic synthesis and cellular proliferation and differentiation.

Without wishing to be bound by theory, the inventor believes that the specific and unique combination of these components present in the compositions and extracts of the invention are responsible for stimulating the production of HA and/or stabilising levels of HA in the extracellular matrix of the skin. It is believed that these components work together in harmony or synergy to provide the beneficial effects, by providing the building blocks for HA production. There is no HA present in the composition or extract itself, but the composition or extract comprises the components needed for stimulating HA production. The composition or extract of the invention therefore offers a gentle and effective means for treating conditions associated with a decline in HA.

It is envisaged that isolated and concentrated extracts comprising one or more of the important bioactive components either alone or in combination with other bioactive components could be produced, which could then be used in various product formulations for more specific and targeted applications.

### Formulations

The compositions or extracts of the invention can be incorporated into a variety of pharmaceutical, cosmeceutical, or nutraceutical formulations or compositions, comprising one or more carriers and/or suitable excipients.

In preferred embodiments of the invention, the composition or an extract derived therefrom, is incorporated into topical formulations in an amount in a range of from about 1% by weight to about 10% by weight. Preferably, the composition or extract is incorporated into such products in an amount in a range from about 1% by weight to about 4% by weight.

In accordance with certain embodiments, various topical cosmetic or pharmaceutical compositions can be provided in the form of an ointment, cream, lotion, gel or other transdermal delivery systems.

Ointments, as used herein, refer to semi-solid preparations including an ointment base having one or more active ingredients incorporated or fused (i.e., melted together with other components of the formulation and cooled with constant stirring to form a congealed preparation) therein. The ointment base may be in the form of: an oleaginous or hydrocarbon base (e.g., petrolatum or a petrolatum/wax combination); an absorption base which permits the incorporation of aqueous solution resulting in the formation of a water-in-oil emulsion (e.g., hydrophilic petrolatum) or which is a water-in-oil emulsion that permits the incorporation of additional quantities of aqueous solutions (e.g., lanolin); a water-removable base which are oil-in-water emulsions that may be diluted with water or aqueous solutions (e.g., hydrophilic ointment, USP); or a water-soluble base that do not contain oleaginous components (e.g., polyethylene glycol (PEG) formulations which combine PEGs having an average molecular below 600 with a PEG having an average molecular weight above 1,000); and the like.

Creams, as used herein, refer to semisolid preparations containing the pollen-based composition or extract of the invention, dissolved or dispersed in either a water-in-oil emulsion or an oil-in-water emulsion or in another type of water-washable base. Generally, creams are differentiated from ointments by the ease with which they are applied/spread onto a surface such as the skin and the ease with which they are removed from a treated surface.

Lotions, as used herein, refer to suspensions of solid materials in an aqueous vehicle. Generally, lotions have a non-greasy character and increased spreadability over large areas of the skin than ointments, creams, and gels.

Gels, as used herein, refer to semisolid systems including a dispersion of small and/or large molecules in an aqueous liquid vehicle which is rendered jellylike by the addition of a gelling agent. Suitable gelling agents include, but are not limited to, synthetic macromolecules (e.g., carbomer polymers), cellulose derivatives (e.g., carboxymethylcellulose and/or hydroxypropyl methylcellulose), and natural gums (e.g., tragacanth gum, carrageenan, and the like). Gel preparations may be in the form of a single-phase gel in which the active or medicinal ingredients are uniformly dispersed throughout the liquid vehicle without visible boundaries or a two-phase gel wherein flocculants or small distinct particles of the active or medicinal ingredient are dispersed within the liquid vehicle.

Transdermal preparations may be formed from an ointment, cream, or gel that has been combined with a penetration enhancer and are designed to deliver an active or medicinal ingredient systemically. Penetration enhancers include, for example, dimethyl sulfoxide, ethanol, propylene glycol, glycerine, PEG, urea, dimethyl acetamide, sodium lauryl sulfate, poloxamers, Spans, Tweens, lecithin, and/or terpenes amongst others.

Other suitable semi-solid forms for use as cosmetic and/or topical pharmaceutical compositions include pastes (preparations containing a larger proportion of solid material rendering them stiffer than ointments) and glycerogelatins (plastic masses containing gelatin, glycerine, water, and an active or medicinal ingredient).

In addition, shampoos or other types of cleansing products are contemplated. Also, topical sprays are contemplated including sprays useful for one or more applications to the skin, back, neck, arms, legs, and torso, for example.

For oral administration, the pollen-based composition or extract derived therefrom, or a solid formulation thereof such as lyophilized powder, may be combined with one or more solid inactive ingredients for the preparation of tablets, capsules, pills, powders, granules or other suitable dosage forms. For example, the composition or extract may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like. It is also possible for oral formulations such as capsules and tablets to include other beneficial bioactive components such as collagen, probiotics, prebiotics, vitamins, minerals, or other components which may provide additional health benefits. In oral formulations, the amount of composition or extract included in the formulation may be much higher, for example, oral capsule formulations may comprise the composition or extract in an amount of at least 20% by weight, or more preferably at least 50% by weight. It is also contemplated that an oral capsule formulation could be produced comprising 100% of the composition or extract of the invention, without any excipients required.

### FORMULATION EXAMPLES

### Example 1 (Topical Skin Cream)

A topical skin cream formulation was prepared as follows:

| | |
|---|---|
| Water | 61.89% |
| Prunus Amygdalus Dulcis (Sweet | 25.00% |
| Almond) oil | |
| Composition or Extract of the Invention | 4.00% |
| Cetearyl Alcohol | 2.00% |
| Glyceryl Stearate SE | 2.00% |
| Cetearyl Olivate | 1.80% |
| Sorbitan Olivate | 1.20% |
| Sodium Stearoyl Glutamate | 1.00% |
| Benzyl Alcohol | 0.70% |
| Xanthum gum | 0.20% |
| Tocopheryl Acetate | 0.10% |
| Dehydroacetic Acid | 0.06% |
| Citric acid | 0.05% |

### Example 2 (Vaginal Cream)

In a further embodiment, a topical vaginal cream formulation was prepared as follows:

| | |
|---|---|
| Water BP | 67.98% |
| Composition or Extract of the Invention | 4.00% |
| Cetearyl Alcohol BP | 10.00% |
| Caprylic Capric Triglycerides USP | 10.00% |
| Polysorbate 60 USP-NF/EP | 3.00% |
| Sorbitan Stearate USP-NF/EP | 2.00% |
| Cetyl palmitate USP-NF | 2.00% |
| Phenoxyethanol | 1.00% |
| Rose Geranium Oil | 0.02% |

### Example 3 (Vaginal Cream)

In a further embodiment, a topical vaginal cream formulation was prepared as follows:

| | |
|---|---|
| Water BP | 63.96% |
| Composition or Extract of the Invention | 4.00% |
| Cetearyl Alcohol BP | 10.00% |
| Caprylic Capric Triglycerides USP | 10.00% |
| Polysorbate 60 USP-NF/EP | 3.00% |
| Butyspermum Parkii (Shea) Butter | 2.00% |
| Sodium Hyaluronate (and) Phenoxyethanol | 2.00% |
| Sorbitan Stearate USP-NF/EP | 2.00% |
| Cetyl palmitate USP-NF | 2.00% |
| Phenoxyethanol | 1.00% |
| Rose Geranium Oil | 0.04% |

### Example 3 (Capsule Formulation)

In a further embodiment, the following capsule formulation was prepared:

| Ingredient | Wt (%) |
|---|---|
| Composition or Extract of the Invention | 56% |
| Lactobacillus Acidophilus | 11.8% |
| Bifidobacterium bifidum 10B/g Powder | 11.8% |
| Excipients | 20.1% |

### Example 4 (Capsule Formulation)

In a further embodiment, the following capsule formulation was prepared:

| Ingredient | Wt (%) |
|---|---|
| Composition or Extract of the Invention | 50% |
| Collagen | 45% |
| Excipients | 5% |

### Example 5 (Lip Balm)

In a further embodiment, the following lip balm formulation was prepared. The formulation was prepared by first melting the beeswax, oils and shea butter. After the mixture was cooled slightly, the flavour was added, and lastly the antioxidant (Vitamin E) was added, and the mixture was mixed well before it was poured into suitable containers and left to set (uncovered to avoid condensation).

| Ingredient | Wt (%) |
|---|---|
| Composition or Extract of the Invention | 3.00% |
| Jojoba oil | 34.00% |
| Almond oil | 30.00% |
| Beeswax | 20.00% |
| Shea butter | 5.00% |
| Argan oil | 4.00% |
| Vanilla flavour | 3.00% |
| Vitamin E | 1.00% |

### Efficacy Studies of Formulations

### Lip Balm Trial

A trial was conducted by an independent supervised university student, to determine the efficacy of the lip balm formulation of Example 4. The participants of the trial included 21 post-menopausal women who would have a propensity towards dry, cracking, thin and pale lips. The participants had all previously been using a standard lip balm product 2-3 times daily for at least 3 months. This was to ensure that any benefits of the product could be attributed to the active ingredient in the product (the extract of the invention) rather than the base ingredients, which are present in most standard lip balm formulations. The participants were asked to complete a survey over a period of 4 weeks, in which they were asked to input a score on a scale of 1 to 5 (1 being the lowest) in relation to four different parameters, namely, whether there were any beneficial changes in hydration, volume, cracking and colour of the lips.

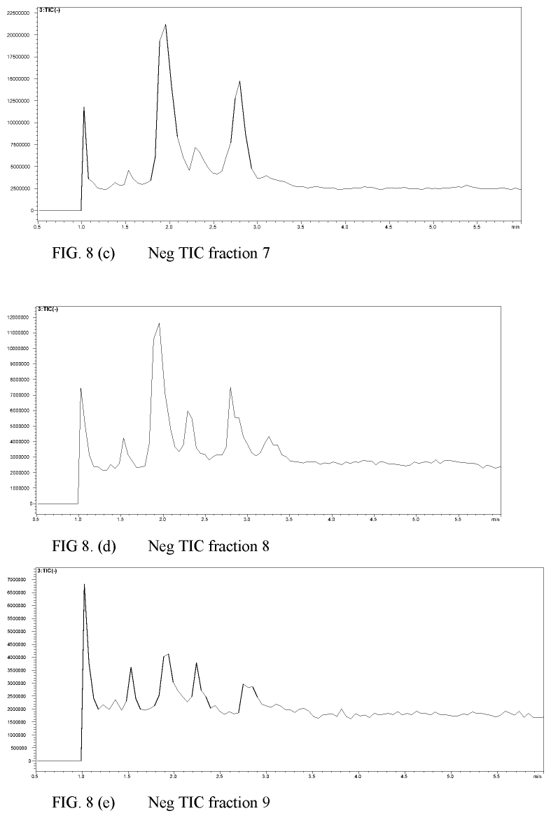

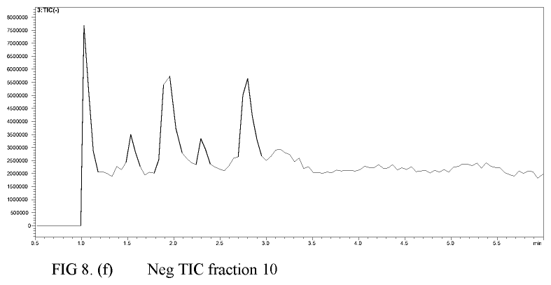

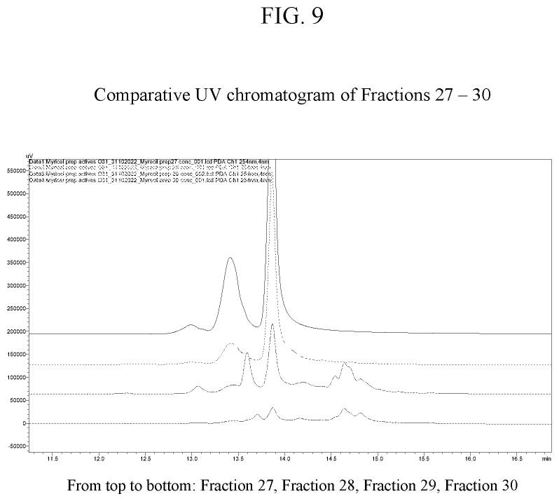

The data from this trial is presented in FIGS. 9 to 12. The data shows that the majority of the participants in the trial recorded an improvement in respect of each of the four parameters tested. After analysing the data and excluding participants that had already reported a 5 in respect of a parameter at the start of the trial, the results showed that a significant majority of participants recorded an improvement in hydration (about 90%). About 60% of participants recorded an improvement in volume, about 77% of participants recorded an improvement in cracking, and about 60% of participants recorded an improvement in colour. These improvements were seen after just two weeks of using the lip balm formulation comprising the extract of the invention.

Given that the skin structure of lips resembles that of vaginal skin tissue, it is expected that formulations comprising the extract of the invention will provide similar benefits when used to treat the symptoms of vaginal atrophy, including vaginal dryness, irritation, redness, and inflammation.

### Vaginal Cream Trial

Since the composition and extract of the invention was found to increase hyaluronic acid in keratinocytes, and to be beneficial for the treatment of lips, a preliminary test of the vaginal cream formulation of Example 2 was carried out in order to test safety, potential efficacy and tolerance of the formulation before commencement of a clinical trial.

The trial was carried out independently by a supervised university student. The participants of the trial included 10 post-menopausal women referred to the trial by a Gynecologist. Each subject was instructed to apply the vaginal cream on the vulva and entrance to the vagina in the evening for a period of two weeks. The participants were asked to complete a survey over those two weeks, in which they were asked to input a score on a scale of 1 to 4 or 5 (1 being no symptoms and 4 or 5 being severe symptoms) in relation to four different parameters, namely, vaginal redness, itching, dryness and thinning/cracking. The survey results together with any comments or feedback were sent directly to the Gynecologist.

The data from this trial is presented in FIGS. 13 to 16. The data shows that the majority of the participants in the trial recorded an improvement in respect of each of the four parameters tested.

Redness was reported on a scale of 1-4. FIG. 13 shows that 50% of participants recorded an improvement over the two weeks. However, 3/10 participants had recorded no redness at week 0, so had no room for improvement. One person reported no symptoms at the start, but then noticed redness in week 1, before returning to no redness in week 2. One person had consistent moderate redness. Excluding those that were reporting a 1 at the start, 5/6 or 83.33% of participants reported an improvement in redness of the vaginal area.

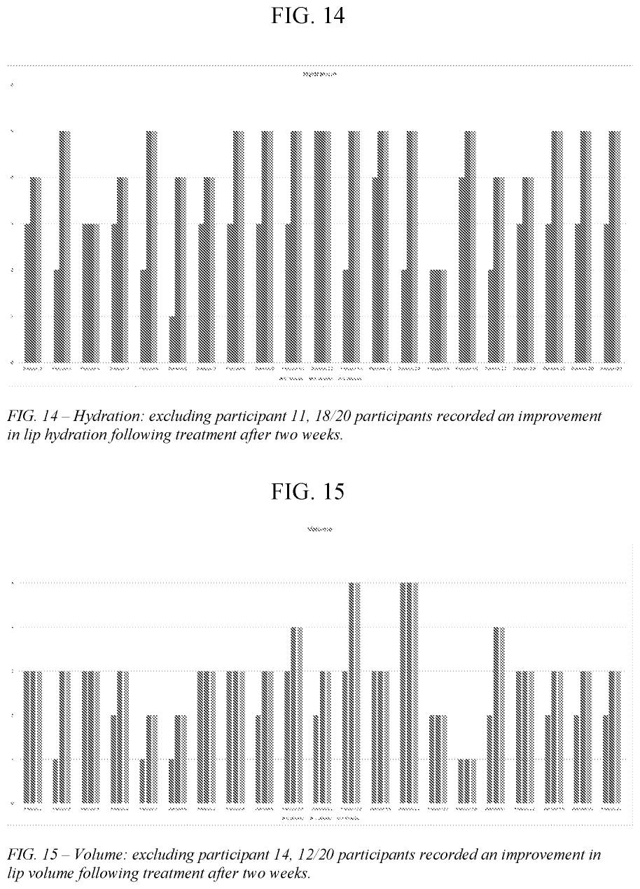

Itching was reported on a scale of 1-5, with 1 being none, and 5 being severe. FIG. 14 shows that 80% of participants reported an improvement in itching. One person reported no itching from the start, so excluding them, the data shows 8/9 or 88.89% had an improvement.

Dryness was reported on a scale of 1-5, with 1 being no dryness and 5 being severe dryness. FIG. 15 shows that all participants reported some improvement in dryness, with 10/10 or 100% of participants dropping at least 1 on the scale.

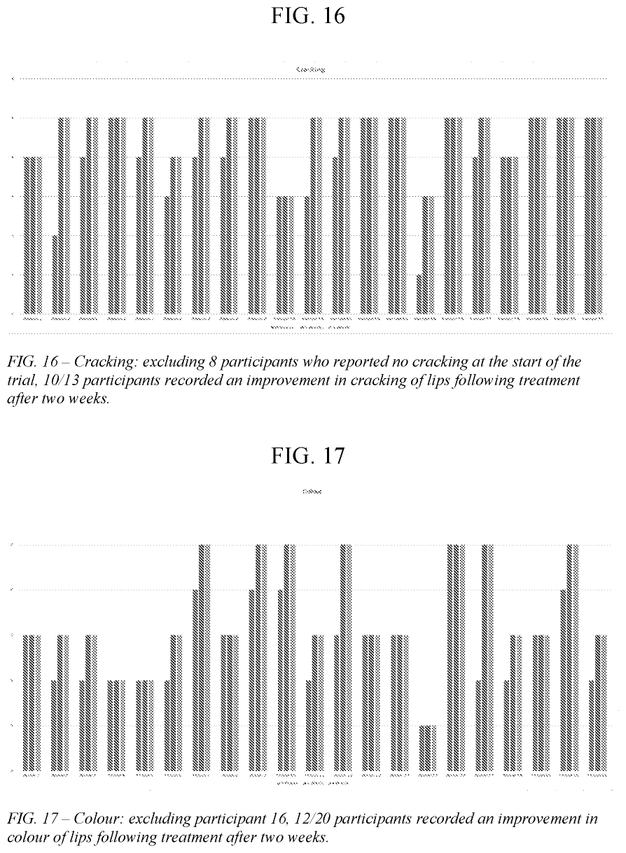

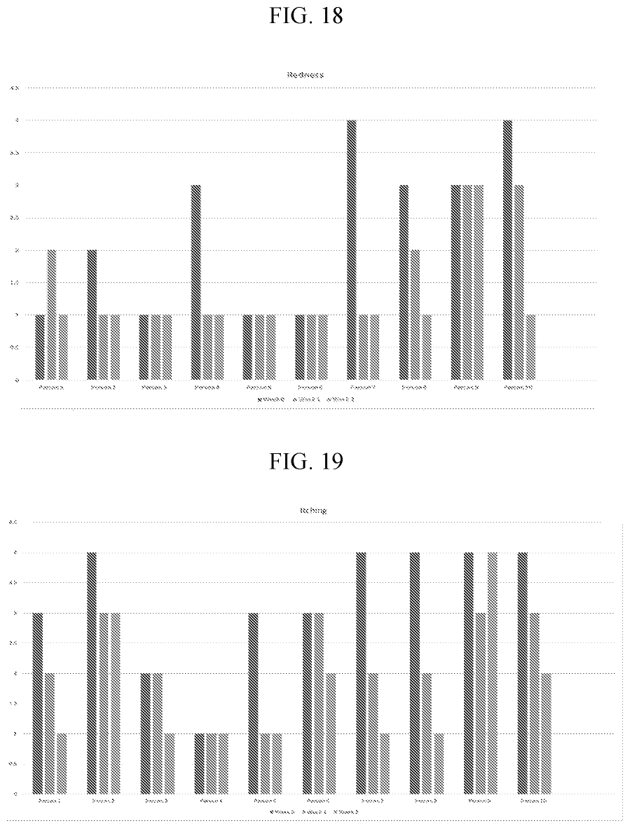

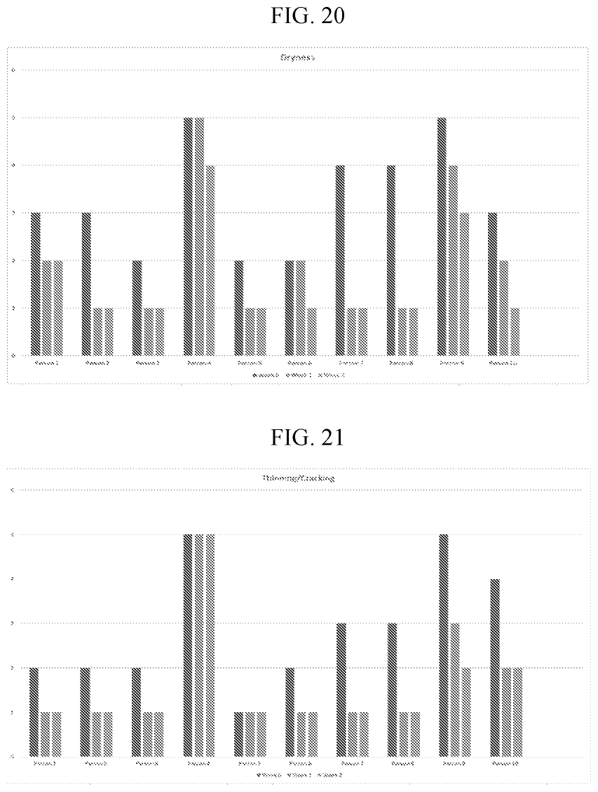

Thinning/cracking was also reported on a scale from 1-5, with 1 being normal and 5 being severe. FIG. 16 shows that 8/10 or 80% of participants reported an improvement. One person reported no thinning or cracking in week 0, so excluding them, 8/9 or 88.89% of participants reported an improvement.

While there was no question in the survey related to bladder strength or urinary incontinence, 6/10 participants provided feedback in the comments section of the survey, or directly to the Gynaecologist, that they noticed a decrease in bladder leakage while using the product, and that their bladder appeared stronger.

It was also reported by 4/10 participants that they had an improved physical response and intensity of orgasm during sexual activity. It is likely that a similar response may be seen in males also, given that the tissues of the penis suffer the same deterioration of HA through the aging process, so it is possible that the composition and extract of the invention could be used on the penis, or orally by males, to increase the production of HA, thereby treating or improving sexual response in males.

These preliminary results show that the composition and extract of the invention is useful for the treatment of vaginal atrophy and the symptoms of GSM. A double-blinded placebo controlled clinical trial of the formulation of the invention will soon be commenced, to establish the efficacy of the formulation in relation to treating GSM in post-menopausal women.

### Advantages

The pollen-based composition and extracts derived therefrom have several potentially realizable advantages. They are natural, gentle formulations. They work by a different, more gentle mechanism of action than current products such as retinol and backuchiol that cause apoptosis and trigger an inflammatory/stress response in skin cells. Consequently, the extracts and compositions of the invention provide a more gentle, long lasting and stabilizing effect on HA levels. They are safe compositions containing no allergenic proteins. The formulations of the invention are easy to incorporate into various product formats. The formulations are effective at treating various skin conditions, including skin aging, as well as the symptoms of GSM, including urinary incontinence, vaginal dryness, vaginal atrophy, irritation, itching, cracking, thinning, redness, inflammation and declining libido, sexual function, sexual response and/or pleasure. It is envisaged that formulations of the invention could also be used to treat other conditions where the stimulation or stabilization of HA is important or helpful.

1. A pollen-based composition, or an extract derived therefrom, wherein the composition or extract comprises at least glucose-6-phosphate, as a substrate or precursor for the production of hyaluronic acid (HA).

2. The composition or extract as claimed in claim 1, wherein the composition or extract further comprises one or more of hexokinases, glucokinases, proteases, peptidases, and magnesium and other elements.

3. The composition or extract as claimed in claim 1, wherein the composition or extract further comprises one or more small molecular weight water-soluble molecules and/or compounds selected from the group comprising anthranilic acid and isomers thereof.

4. The composition or extract as claimed in claim 1, wherein the composition or extract further comprises one or more compounds selected from the group comprising flavonoids, flavonols, flavones, alkaloids, aglycones, glycosides, lipids, fatty acids, spermidine and isomers thereof, caffeoyl spermidine compounds, feruloyl spermidine compounds and other compounds that are cinnamic acid conjugates of spermidine, and hydroxycinnamic acids.

5. The composition or extract as claimed in claim 4, wherein the caffeoyl spermidine compounds are selected from one or more of the group comprising tricaffeoyl-spermidine (N1,N5,N10-tricaffeoyl spermidine), triferuloyl-spermidine (N1,N5,N10-Tri-feruloyl spermidine), and dicaffeoyl-p-coumaroyl-spermidine.

6. The composition or extract as claimed in claim 4, wherein the hydroxycinnamic acids are selected from the group comprising dicaffeoylquinic acids and dicaffeoyl tartaric acid and isomers thereof.

7. The composition or extract as claimed in claim 4, wherein the flavonoids, flavonols and/or flavones are selected from the group comprising isorhamnetin, rhamnetin, tricetin, and kaempferol.

8. The composition or extract as claimed in claim 4, wherein the lipids are selected from the group comprising fatty acids, phospholipids, cholesterol and glycolipids.

9. The composition or extract as claimed in claim 8, wherein the fatty acids are selected from the group comprising myristic acid, palmitic acid, linoleic acid, and stearic acid.

10. The composition or extract as claimed in claim 1, wherein the composition is prepared by a method comprising at least the following steps:
a. Combining one or more of raw bee pollen, raw honey and raw propolis with one or more of coconut water, coconut oil and/or coconut cream, to form a mixture;
b. Anaerobically incubating the mixture at a temperature of between about 25° C. to 65° C. for a period of between about 12 to 96 hours; and
c. Freezing and/or freeze drying the resulting fermented composition.

11. The composition or extract as claimed in claim 10, further comprising an additional step of concentrating the resulting fermented composition prior to step (c).

12. The composition or extract as claimed in claim 11, wherein the concentration step comprises centrifugation and collection of the supernatant for freezing and/or freeze-drying.

13. The composition or extract as claimed in claim 10, wherein the composition or extract is a freeze-dried powdered composition or extract.

14. A formulation comprising the pollen-based composition or extract as claimed in claim 1, together with one or more carriers and/or excipients.

15. The formulation as claimed in claim 14, wherein the formulation is a topical formulation comprising between about 1-10% by weight of the pollen-based composition or extract.

16. The formulation as claimed in claim 14, wherein the formulation is an oral formulation comprising at least 20% by weight of the pollen-based composition or extract.

17. The formulation as claimed in claim 14, wherein the formulation is for treating or preventing one or more symptoms of Genitourinary Syndrome of Menopause (GSM), including one or more of urinary incontinence, vaginal atrophy, vaginal dryness, irritation, itching, inflammation, lack of sexual response and lack of libido.

18. A method of stimulating the production of hyaluronic acid (HA) in a human subject, said method comprising administering to the subject a therapeutically effective amount of a formulation as claimed in claim 14.

19. A method of treating or preventing a condition associated with a decline in hyaluronic acid production or degradation of hyaluronic acid in a human subject, said method comprising administering to the subject a therapeutically effective amount of a formulation as claimed in claim 14.

20. A method for the treatment or prevention of a condition in a human subject, said condition selected from the group comprising skin disorders, skin aging, Genitourinary Syndrome of Menopause (GSM) including urinary incontinence, vaginal dryness, vaginal atrophy, irritation, itching, cracking, thinning, redness, inflammation and declining libido, sexual function, sexual response and/or pleasure, dry eyes, connective tissue and joint disorders, and other conditions that may benefit from the stimulation or stabilization of hyaluronic acid (HA), said method comprising administering to the subject a therapeutically effective amount of a formulation as claimed in claim 1.
